(19) United States
(12) Patent Application Publication
(10) Pub. No.: US 2003/0190334 A1
(43) Pub. Date: Oct. 9, 2003

(54) Low dose liquid entecavir formulations and use

(76) Inventors: Divyakant Desai, West Windsor, NJ (US); Danping Li, East Brunswick, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/407,287

(22) Filed: Apr. 4, 2003

Related U.S. Application Data
(60) Provisional application No. 60/370,674, filed on Apr. 8, 2002.

(51) Int. Cl. A61K 31/522

(52) U.S. Cl. 424/400; 514/263.37

(57) ABSTRACT

Liquid pharmaceutical compositions are provided having a low dose of entecavir. In one embodiment of the present invention, the liquid entecavir composition is a ready-to-use composition that is formulated to be both stable and palatable. In a second embodiment of the present invention, the liquid entecavir composition is formulated from a powder composition at the time of use. The low dose entecavir compositions may also include at least one component selected from sweetener, preservative, flavoring agent, buffering agent, or combinations thereof. The liquid entecavir compositions may also be formulated in combination with other pharmaceutically active agents.

This application claims priority from Serial No. 60/370,674 filed Apr. 8, 2002.

### BACKGROUND OF THE INVENTION

Entecavir, [1S-(1α,3α,4β)]-2-amino-1,9-dihydro-9-[4-hydroxy-3-(hydroxymethyl)-2-methylenecyclopentyl]-6H-purin-6-one, having the chemical structure

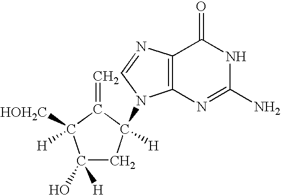

is an antiviral agent currently undergoing clinical evaluation for the treatment of hepatitis B virus (HBV) infection.

U.S. Pat. No. 5,206,244 to Zahler et al., discloses entecavir and its use in treating hepatitis B. Zahler discloses that an effective antiviral dose for oral or parenteral administration will likely be in the range of about 1.0 to 50 mg/kg of body weight and that the desired dose may be administered several times daily at appropriate intervals.

Improved methods for the synthesis of entecavir are disclosed by Bisacchi et al. in WO 98/09964.

Low dose entecavir formulations, particularly tablets and capsules, are disclosed in U.S. application US-2001-0033864-A1 published Oct. 25, 2001 and PCT application WO 01/64221 A1 published Sept. 7, 2001.

### SUMMARY OF THE INVENTION

It is an object of the present invention to provide a liquid pharmaceutical composition having a low dose of entecavir capable of safely and effectively treating hepatitis B virus infection.

It is another object of the present invention to provide such a low dose liquid entecavir composition that is ready-to-use.

It is still another object of the present invention to provide such a low dose liquid entecavir composition that is both stable and palatable.

It is yet another object of the present invention to provide such a low dose liquid entecavir composition that is formulated from a powder for constitution as a liquid composition at the time of use.

It is a further object of the present invention to provide a process for formulating a low dose, ready-to-use, liquid entecavir composition.

It is still a further object of the present invention to provide a process for formulating a low dose, liquid entecavir composition from a powder for constitution as a liquid composition at the time of use.

These and other objects and advantages of the present invention are accomplished by a liquid pharmaceutical composition having a low dose of entecavir. In one embodiment of the present invention, the liquid entecavir composition is a ready-to-use composition that is formulated to be both stable and palatable. In a second embodiment of the present invention, the liquid entecavir composition is formulated from a powder for constitution as a liquid composition at the time of use. The low dose entecavir compositions also have at least one additional component selected from one or more of the following: sweetener, preservative, flavoring agent, buffering agent, pH adjusting agent, or any combinations thereof. The liquid entecavir compositions may also be formulated in combination with other pharmaceutically active agents.

### DETAILED DESCRIPTION OF THE INVENTION

Entecavir is a potent antiviral agent, which has shown good efficacy against HBV. Since entecavir is very potent, very low doses are sufficient to achieve the desired therapeutic effects. However, low dose formulations, and notably liquid formulations, pose great challenges to formulators because the drug degrades faster in the liquid state than in the solid state. Any minor degradation translates into a significant reduction in potency.

While entecavir is potent, it is also extremely bitter. To combat the bitterness, a sweetener is generally used. However, entecavir has shown a tendency to react with commonly used sweeteners, such as sucrose, creating stability concerns. Entecavir has a primary amine group in its structure that has the propensity to react with any sweetener or flavoring agent that contains aldehyde and/or ketone groups. This reaction is more pronounced at a weakly acidic pH (pH 3 to 4), but minimized at pH 5 to 7.

This invention is directed to liquid pharmaceutical compositions containing a low dose of the active antiviral agent entecavir for once daily administration to treat hepatitis B virus infection in an adult human patient or a pediatric patient. The liquid pharmaceutical compositions also have at least one additional component selected from one or more of the following: sweetener, preservative, flavoring agent, buffering agent, pH adjusting agent, other pharmaceutically active agents particularly another antiviral agent, or any combinations thereof.

The term adult human patient is defined as a patient of about 16 years or more of age and a weight equal to or greater than about 50 kilograms. Pharmaceutical compositions containing entecavir at the lower end of the above ranges are suitable for administration to pediatric patients or adult patients weighing less than about 50 kilograms.

In one embodiment of the present invention, the liquid entecavir composition is a ready-to-use pharmaceutical composition. The concentration of each component present in the liquid entecavir composition is reflected in a percent weight by volume (% w/v). The antiviral agent entecavir is present in the liquid, ready-to-use pharmaceutical composition in an amount about 0.001% to about 20%. Preferably, the entecavir is present in the composition in an amount about 0.003% to about 10%, more preferably between about 0.005% and about 5%, and most preferably between about 0.005% and about 1%.

To counteract the bitterness associated with entecavir, and to make the composition palatable, a sweetener may be added to the composition. Suitable sweeteners include, for example, maltitol (Lycasin®), sucrose, sorbitol, xylitol, mannitol, or any combinations thereof. The sweetener is present in the composition in an amount about 10% to about 85%. Preferably, sweetener is present in an amount about 15% to about 70%.

To further enhance the palatability of the entecavir composition of the present invention, a flavoring agent may be added to the composition. Suitable flavoring agents include, for example, cherry, guarana, orange, banana, strawberry, vanilla, chocolate, or any combinations thereof. The flavoring agent may be present in the composition in an amount about 0.001% to about 2%. Preferably, the flavoring agent is present in an amount about 0.01% to about 0.075%.

The composition of the present invention may also include a preservative. Suitable preservatives include, for example, methylparaben, propylparaben, butylparaben, sodium benzoate, potassium sorbate, or any combinations thereof. The preservative may be present in the composition in an amount about 0.01% to about 1.0%. Preferably, preservative is present in an amount about 0.1% to about 0.75%.

The pH of the composition may be adjusted with any suitable dilute acid or base. By way of example, a suitable dilute acid is hydrochloric acid and a suitable dilute base is sodium hydroxide. The pH of the composition is preferably about 5 to about 7.

The inclusion of a buffering agent to maintain a composition pH of about 5 to about 7 is important both for the stability of the entecavir with the sweetener and the stability of the preservative. Suitable buffering agents include, for example, citric acid, sodium citrate, phosphate buffer, acetate buffer, or any combinations thereof. The buffering agent is present in the composition in an amount sufficient to maintain a composition pH of about 5 to about 7. Preferably, the molar concentration of the buffering agent is between about 5 mM to about 200 mM. The buffering agent is present in the composition in an amount about 0.01% to about 5%.

The above components of the liquid entecavir composition may be formulated in solution with any suitable pharmaceutically acceptable solvent. A suitable pharmaceutically acceptable solvent includes, for example, water, PEG 400, propylene glycol, ethanol, glycerin, or any combinations thereof. Preferably, the pharmaceutically acceptable solvent is water.

Two preferred, ready-to-use, liquid entecavir compositions of the present invention are set forth in Tables 1 and 2 below.

TABLE 1
Ready to Use (RTU) Entecavir (0.2 mg/mL) Liquid Formulation

| Ingredients | Gram/100 ml | Function |
|---|---|---|
| Entecavir | 0.02 | Antiviral Agent |
| Maltitol (Lycasin ®) | 65.0 | Sweetener |
| Methylparaben | 0.2 | Preservative |
| Propylparaben | 0.028 | Preservative |
| Cherry or Guarana | 0.05/0.025/0.025 | Flavoring Agent |
| or Orange | | |
| Citric Acid/Sodium Citrate | 0.96/1.47 (100 mM) or | Buffering Agent |
| | 0.037/0.24 (10 mM) | |
| Water | q.s. to 100 mL (pH 6.0) | Solvent |

TABLE 2
Ready to Use (RTU) Entecavir (0.05 mg/mL) Liquid Formulation

| Ingredients | Gram/100 ml | Function |
|---|---|---|
| Entecavir | 0.005 | Antiviral Agent |
| Maltitol (Lycasin ®) | 65.0 | Sweetener |
| Methylparaben | 0.2 | Preservative |
| Propylparaben | 0.028 | Preservative |
| Cherry or Guarana | 0.05/0.025/0.025 | Flavoring Agent |
| or Orange | | |
| Citric Acid/Sodium Citrate | 0.96/1.47 (100 mM) or | Buffering Agent |
| | 0.037/0.24 (10 mM) | |
| Water | q.s. to 100 mL (pH 6.0) | Solvent |

The stability of the ready-to-use liquid entecavir compositions of the present invention is demonstrated below in Tables 3 and 4.

TABLE 3
Stability of Entecavir Ready-to-Use (RTU) Liquid Formulation^{a}, 0.05 mg/mL, in Clear Glass Bottles

| | | | | Impurity/ | | | | | | |
|---|---|---|---|---|---|---|---|---|---|---|
| | | Entecavir | | Degradant, % I.I. | | Methylparaben | | Propylparaben | | |
| Time, | Storage | Potency, | | | | Potency, | | Potency, | | pH/ |
| weeks | condition | mg/mL | % Label | RRT 0.24 | Total | mg/mL | % Label | mg/mL | % Label | Appearance |
| Initial | | 0.0508 | 101.6 | 0.07 | 0.07 | 1.94 | 97 | 0.273 | 97.5 | 5.99/complies |
| 4 days | 25° C./HIL/UVA^{b}, PROT | 0.0506 | 101.2 | 0.07 | 0.07 | 1.98 | 99 | 0.277 | 98.9 | 6.02/complies |
| | 25° C./HIL/UVA^{b}, EXPOS | 0.0503 | 100.6 | 0.07 | 0.07 | 1.98 | 99 | 0.278 | 99.3 | 6.03/complies |
| 2 | 25° C./HIL/UVA^{b} PROT | 0.0506 | 101.2 | 0.07 | 0.07 | 2.00 | 100 | 0.284 | 101.4 | 5.67/complies |
| | 25° C./HIL/UVA^{b} EXPOS | 0.0499 | 99.8 | 0.07 | 0.74^{c} | 1.98 | 99.0 | 0.279 | 99.6 | 5.91/complies |
| | 30° C./60% RH | 0.0512 | 102.4 | 0.06 | 0.06 | 2.0 | 100 | 0.281 | 100.4 | 6.02/complies |
| | 40° C./75% RH | 0.0513 | 102.6 | 0.09 | 0.09 | 1.99 | 99.5 | 0.278 | 99.3 | 6.03/complies |
| 4 | 30° C./60% RH | 0.0509 | 101.8 | 0.07 | 0.07 | 1.97 | 98.5 | 0.280 | 100 | 6.03/complies |
| | 40° C./75% RH | 0.0509 | 101.8 | 0.07 | 0.07 | 1.95 | 97.5 | 0.279 | 99.6 | 6.01/complies |
| | 50° C. | 0.0505 | 101.0 | 0.08 | 0.08 | 1.89 | 94.5 | 0.276 | 98.6 | 6.00/complies |
| 13 | 25° C./60% RH | 0.0509 | 101.8 | 0.07 | 0.07 | 2.00 | 100 | 0.286 | 102.1 | 6.03/complies |
| | 30° C./60% RH | 0.0515 | 103.0 | 0.07 | 0.07 | 1.97 | 98.5 | 0.282 | 100.7 | 6.01/complies |
| | 40° C./75% RH | 0.0508 | 101.6 | 0.07 | 0.07 | 1.91 | 95.5 | 0.281 | 100.4 | 6.00/complies |
| | 50° C. | 0.0500 | 100.0 | 0.05 | 0.05 | 1.73 | 86.5 | 0.272 | 97.1 | 5.99/complies |
| 26 | 5° C. | 0.0512 | 102.4 | <0.05 | <0.05 | 2.00 | 100 | 0.284 | 101.4 | 5.99/complies |
| | 25° C./60% RH | 0.0509 | 101.8 | <0.05 | <0.05 | 1.96 | 98 | 0.281 | 100.4 | 6.00/complies |
| | 30° C./60% RH | 0.0510 | 102 | <0.05 | <0.05 | 1.94 | 97 | 0.280 | 100.0 | 5.99/complies |
| | 40° C./75% RH | 0.0501 | 100.2 | <0.05 | <0.05 | 1.80 | 90 | 0.274 | 97.9 | 5.99/complies |

^{a}The solutions were stored in 4 oz (120 mL) glass bottles with child-resistant cap at 66-mL fill. The bottles were stored at an upright position except for photostability samples, where the bottles were stored lying on their side.
^{b}HIL/UVA = Ultraviolet-A (range 320-400 nm) and high-intensity visible fluorescent light.
The ICR guidelines of 200 watt hours/m² and 1.2 × 10⁶ lux hours are met after approximately 4 days.
^{c}Two additional degradants with RRT of 1.8 (0.61% I.I.) and 2.1 (0.05% I.I.) were detected in this sample.
RH = relative humidity
RRT = relative retention time
PROT = protected (bottle has cap)
EXPOS = unprotected (no cap)

TABLE 4
Stability of Entecavir Ready-to-Use (RTU) Liquid Formulation^{a}, 0.2 mg/mL, in Clear Glass Bottles

| | | | | Impurity/ | | | | | | |
|---|---|---|---|---|---|---|---|---|---|---|
| | | Entecavir | | Degradant, % I.I. | | Methylparaben | | Propylparaben | | |
| Time, | Storage | Potency, | | | | Potency, | | Potency, | | pH/ |
| weeks | condition | mg/mL | % Label | RRT 0.24 | Total | mg/mL | % Label | mg/mL | % Label | Appearance |
| Initial | | 0.204 | 102 | 0.07 | 0.07 | 1.87 | 93.5^{c} | 0.264 | 94.3^{c} | 5.96/complies |
| 4 days | 25° C./HIL/UVA^{b}, PROT | 0.201 | 100.5 | 0.07 | 0.07 | 1.96 | 98 | 0.277 | 98.9 | 6.02/complies |
| | 25° C./HIL/UVA^{b}, EXPOS | 0.200 | 100 | 0.06 | 0.06 | 1.95 | 97.5 | 0.275 | 98.2 | 6.04/complies |
| 2 | 25° C./HIL/UVA^{b} PROT | 0.203 | 101.5 | 0.07 | 0.07 | 1.99 | 99.5 | 0.282 | 100.7 | 5.75/complies |
| | 25° C./HIL/UVA^{b} EXPOS | 0.199 | 99.5 | 0.07 | 0.26^{d} | 1.97 | 98.5 | 0.278 | 99.3 | 5.94/complies |
| | 30° C./60% RH | 0.205 | 102.5 | 0.07 | 0.07 | 2.00 | 100 | 0.281 | 100.4 | 5.99/complies |
| | 40° C./75% RH | 0.203 | 101.5 | 0.07 | 0.07 | 1.99 | 99.5 | 0.286 | 102.1 | 6.00/complies |
| 4 | 30° C./60% RH | 0.205 | 102.5 | 0.07 | 0.07 | 1.97 | 98.5 | 0.280 | 100 | 5.99/complies |
| | 40° C./75% RH | 0.203 | 101.5 | 0.08 | 0.08 | 1.95 | 97.5 | 0.280 | 100 | 5.97/complies |
| | 50° C. | 0.204 | 102 | 0.06 | 0.06 | 1.91 | 95.5 | 0.280 | 100 | 5.96/complies |
| 13 | 25° C./60% RH | 0.205 | 102.5 | 0.07 | 0.15^{e} | 2.09 | 104.5 | 0.299 | 106.8 | 6.00/complies |
| | 30° C./60% RH | 0.205 | 102.0 | 0.08 | 0.15^{e} | 2.10 | 105 | 0.301 | 107.5 | 6.00/complies |
| | 40° C./75% RH | 0.205 | 102.5 | 0.07 | 0.07 | 1.89 | 94.5 | 0.279 | 99.6 | 6.03/complies |
| | 50° C. | 0.203 | 101.5 | 0.07 | 0.14^{f} | 1.68 | 84.0 | 0.265 | 94.6 | 5.97/complies |
| 26 | 5° C. | 0.206 | 103 | <0.05 | <0.05 | 1.99 | 99.5 | 0.284 | 101.4 | 5.94/complies |
| | 25° C./60% RH | 0.206 | 103 | <0.05 | <0.05 | 1.97 | 98.5 | 0.283 | 101.1 | 5.94/complies |
| | 30° C./60% RH | 0.204 | 102 | <0.05 | <0.05 | 1.94 | 97 | 0.280 | 100 | 5.99/complies |
| | 40° C./75% RH | 0.202 | 101 | <0.05 | <0.05 | 1.81 | 90.5 | 0.276 | 98.6 | 5.99/complies |

^{a}The solutions were stored in 4 oz (120 mL) glass bottles with child-resistant cap at 66-mL fill. The bottles were stored at an upright position except for photostability samples, where the bottles were stored lying on their side.
^{b}HIL/UVA = Ultraviolet-A (range 320-400 nm) and high-intensity visible fluorescent light.
The ICH guidelines of 200 watt hours/m² and 1.2 × 10⁶ lux hours are met after approximately 4 days.
^{c}The low preservative concentrations in the initial sample was due to assay problem. This was supported by the 98-100% results for the samples stored under the accelerated conditions.
^{d}An additional degradant with RRT of 1.8 (0.19% I.I.) was found in this sample.
^{e}An additional degradant with RRT of 2.33 was found at 0.07% and 0.08% I.I. level in the 25° C./60% RH and 30° C./60% RH samples, respectively.

Clearly, the data set forth above demonstrates that the liquid, ready-to-use entecavir compositions are extremely stable over an extended period of time at varying temperatures, even with the inclusion of a sweetener.

Liquid formulations containing from about 0.001 mg to about 10 mg of entecavir per mL are prepared according to the following procedures that ensure high potency and good uniformity of the product. The ready-to-use liquid compositions are prepared by first carefully dissolving preservatives and entecavir in water. The preservatives and entecavir are dissolved by stirring the solution with heating at a temperature about 40° C. to about 80° C.

Once the preservatives and entecavir are dissolved, sweetener is added to the above solution. The solution is mixed with a mixer at a speed sufficient to form a vortex until the sweetener is dissolved.

After cooling the solution to below 35° C., one or more buffering agents and a flavoring agent are then added to the solution. The solution is mixed with any suitable mixer until both the buffering agent and the flavoring agent are dissolved. If necessary, the pH of the solution may be adjusted to about 5 to about 7 with a diluted acid or base. After adjusting the pH, the remaining water is added to make up the final volume of the batch. The final solution is mixed until uniform. The solution is bottled and stored at room temperature.

In a second embodiment of the present invention, the liquid entecavir composition is formulated from a powder for constitution as a liquid composition at the time of use. With a powder for constitution, the powder is mixed with a predetermined amount of water to form the liquid entecavir composition. One advantage to using a powder is that the stability of the powder can be maintained throughout its shelf life. The concentration of each component of the powder compositions of the present invention is reflected as a weight percent (wt. %) based on the total weight of the powder composition.

The antiviral agent entecavir is present in the powder composition in an amount about 0.001% to about 20%. Preferably, the entecavir is present in the powder composition in an amount about 0.003% to about 10%, more preferably about 0.005% to about 5%, and most preferably about 0.005% to about 1%.

To overcome the bitterness associated with entecavir, and to make the composition palatable at the time of use, a sweetener, as set forth above for the liquid entecavir compositions, may be added to the powder composition. The sweeteners include, for example, sucrose, glucose, acesulfame, dextrose, sorbitol, xylitol, mannitol, or any combinations thereof. The sweetener is present in the powder composition in an amount about 30% to about 98%, based on the total weight of the powder composition. Preferably, sweetener is present in the composition in amount about 60% to about 95%.

To further enhance the palatability of the powder entecavir composition of the present invention, a flavoring agent, such as those set forth above for the liquid compositions, may be added to the powder for constitution composition. The flavoring agents include, for example, cherry, guarana, orange, banana, strawberry, vanilla, chocolate, or any combinations thereof. The flavoring agent may be included in the powder composition in an amount about 0.001 wt. % to about 1 wt. %. Preferably, flavoring agent is present in an amount about 0.01 wt. % to about 0.50 wt. %.

The powder for constitution composition of the present invention may also include a preservative, such as those set forth above for the liquid compositions of the present invention. The preservatives may include, for example, methylparaben, propylparaben, sodium benzoate, potassium sorbate, or any combinations thereof. The preservative may be present in the powder composition in an amount about 0.01 wt. % to about 5 wt. %. Preferably, preservative is present in an amount about 0.50 wt. % to about 3 wt. %.

The inclusion of a buffering agent to maintain a composition pH of about 5 to about 7 is important both for the stability of the entecavir powder composition with the sweetener and the preservative stability. Suitable buffering agents, such as those set forth above for the liquid entecavir compositions may be used. These buffering agents include, for example, citric acid, sodium citrate, phosphate buffer, acetate buffer, or any mixtures thereof. The buffering agent is included in the powder for constitution composition in an amount sufficient to maintain a composition pH of about 5 to about 7. Preferably, the molar concentration of the buffering agent is about 5 mM to about 200 mM. The buffering agent is present in the powder composition in an amount about 1% to about 20%. Preferably, it is present in an amount about 5% to about 15%.

Three preferred liquid entecavir compositions formulated from a powder for constitution of the present invention are set forth in Tables 5 through 7 below.

TABLE 5
Powder for Constitution Entecavir (0.2 mg/mL) Liquid Formulation

| Ingredients | Gram/100 ml | Function |
|---|---|---|
| Entecavir | 0.02 | Antiviral Agent |
| Xylitol | 35.0 | Sweetener |
| Sodium Benzoate | 0.4 | Preservative |
| Cherry or Guarana | 0.05/0.025 | Flavoring Agent |
| Potassium Phosphate, | 2.58/0.04 | Buffering Agent |
| monobasic (KH₂PO₄) | | |
| and Sodium Phosphate, | | |
| dibasic (Na₂HPO₄) | | |
| Purified Water | q.s. to 100 mL (pH 5.0) | Solvent |

TABLE 6
Powder for Constitution Entecavir (0.05 mg/mL) Liquid Formulation

| Ingredients | Gram/100 ml | Function |
|---|---|---|
| Entecavir | 0.005 | Antiviral Agent |
| Xylitol | 35.0 | Sweetener |
| Sodium Benzoate | 0.4 | Preservative |
| Cherry or Guarana | 0.05/0.025 | Flavoring Agent |
| Potassium Phosphate, | 2.58/0.04 | Buffering Agent |
| monobasic (KH₂PO₄₎ | | |
| and Sodium Phosphate, | | |
| dibasic (Na₂HPO₄) | | |
| Purified Water | q.s. to 100 mL (pH 5.0) | Solvent |

TABLE 7
Powder for Constitution Entecavir (0.2 mg/mL) Liquid Formulation

| Ingredients | Gram/100 ml | Function |
|---|---|---|
| Entecavir | 0.02 | Antiviral Agent |
| Mannitol | 15.0 | Sweetener |
| Sodium Benzoate | 0.4 | Preservative |
| Cherry or Guarana | 0.05/0.025 | Flavoring Agent |
| Potassium Phosphate, | 2.58/0.04 | Buffering Agent |
| monobasic (KH₂PO₄) | | |
| and Sodium Phosphate, | | |
| dibasic (Na₂HPO₄) | | |
| Purified Water | q.s. to 100 mL (pH 5.0) | Solvent |

The stability of the liquid entecavir compositions formulated from powders for constitution is demonstrated below in Tables 8 and 9.

TABLE 8
Stability of Powder for Constitution Entecavir (0.2 mg/mL)
Liquid Formulation given in Table 5

| | | In 100 mM Citric Buffer | | |
|---|---|---|---|---|
| | Storage | % of initial potency | | |
| Time | Condition | With Cherry | With Guarana | pH for both flavors |
| 3 weeks | 5° C. | ND | 103 | 5.0 |
| | 40° C. | ND | 101.5 | 5.0 |
| | 50° C. | ND | 103 | 5.0 |

ND—Not determined

TABLE 9
Stability of Powder for Constitution Entecavir (0.05 mg/mL)
Liquid Formulation given in Table 6

| | | In 100 mM Citric Buffer | | |
|---|---|---|---|---|
| | Storage | % of initial potency | | |
| Time | Condition | With Cherry | With Guarana | pH for both flavors |
| 6 weeks | 5° C. | 100 | 100 | 5.0 |
| | 30° C. | 100 | 100 | 5.0 |
| | 40° C. | 104 | 98 | 5.0 |
| | 50° C. | 91 | 80 | 5.0 |

Clearly, the data set forth above demonstrates that the liquid entecavir compositions formed from a powder for constitution composition are extremely stable over an extended period of time at varying temperatures, even with the inclusion of sweetener.

The powder for constitution, entecavir liquid compositions of the present invention may be made by first formulating a powder composition. The powder composition is formed by mixing the entecavir, sweetener, preservative, flavoring agent, and buffering agent in a mixer or blender at a slow speed using geometric mixing. The resulting blend is subdivided in a wide mouth 100-mL bottle. Each bottle will have about 38 grams of blend. To form a liquid composition, the blend is constituted with an amount of water suitable to obtain the desired solution concentration of entecavir.

It should be understood that while the above procedures are described for preparing pharmaceutical compositions containing from about 0.05 mg to about 0.2 mg of entecavir, they can also be employed to prepare pharmaceutical compositions containing low doses of any soluble pharmaceutically active substance.

In another embodiment of the present invention, a hepatitis B virus infection may be treated with low dose entecavir liquid compositions as described above in combination with one or more additional pharmaceutically active agents.

Suitable additional pharmaceutically active agents for this purpose include one or more antiviral agents, for example, didanosine, lamivudine, abacavir, adefovir, adefovir dipivoxil, famciclovir, (2R,4R)-4-(2,6-diamino-9H-purin-9-yl)-2-hydroxymethyl-1,3-dioxolane (DAPD), hepatitis B immunomodulating proteins (EHT 899 from Enzo Biochem), emtricitabine, 1-(2-deoxy-2-fluoro-β-D-arabinofuranosyl)thymine(FMAU), GLQ-223 (Compound A, alpha-trichosanthin), epavudine (L-dT), epcitabine (L-dC), ribavirin, tenofovir (PMPA), 2′,3′-dideoxy-2′,3′-didehydro-beta-L(-)-5-fluorocytidine[L(-)Fd4C], as well as other fluoro L- and D-nucleosides.

Suitable pharmaceutically active agents for this purpose may also include one or more immunomodulators, for example, alpha interferon, beta interferon, pegylated interferon, thymosin alpha, and hepatitis B vaccines such as HBV/MF59, Hepagene and Theradigm-HBV.

In another embodiment of the present invention, co-infected patients may be treated with the low dose liquid entecavir compositions described above. A co-infected patient is one infected with other viral or non-viral diseases in addition to hepatitis B. In particular, such treatment is possible for hepatitis B patients co-infected with hepatitis C or HIV. Such co-infected patients are preferably treated with the low dose liquid entecavir compositions as described above in combination with one or more other pharmaceutically active agents as described above. For example, a patient co-infected with hepatitis B and hepatitis C can be treated with the low dose liquid entecavir composition in addition to being treated with a regimen of ribavirin and an interferon.

The low dose liquid entecavir pharmaceutical compositions described above for daily administration may also be administered to certain patients less often. For example, patients who have been treated by daily administration of the low dose entecavir pharmaceutical compositions so that their hepatitis B virus infection is now under control may be placed on a maintenance regimen to protect against further infection. Such maintenance therapy may involve the administration of the low dose liquid entecavir composition on a less than daily basis. For example, a single dose administered every three or four days or administered on a weekly basis may be sufficient.

Surprisingly, it has been found that once daily administration of the low dose liquid entecavir pharmaceutical compositions of this invention are effective in treating hepatitis B virus infection without undesirable side effects that can result from administration of the high dose regimen described in U.S. Pat. No. 5,206,244.

The present invention having been thus been described with particular reference to the preferred forms thereof, it will be obvious that various changes and modifications may be made therein without departing from the spirit and scope of the present invention as defined in the appended claims.

What is claimed is:

1. A liquid pharmaceutical composition to treat hepatitis B virus infection comprising a pharmaceutically acceptable solvent and about 0.001% to about 20% w/v of entecavir.

2. The liquid pharmaceutical composition of claim 1, wherein said entecavir is present in the composition in an amount of about 0.003% to about 10% w/v.

3. The liquid pharmaceutical composition of claim 2, wherein said entecavir is present in the composition in an amount of about 0.005% to about 5% w/v.

4. The liquid pharmaceutical composition of claim 3, wherein said entecavir is present in the composition in an amount of about 0.005% to about 1% w/v.

5. The liquid pharmaceutical composition of claim 1, further comprising at least one component selected from the group consisting of sweetener, preservative, flavoring agent, buffering agent, pH adjusting agent, pharmaceutically active agent other than entecavir, and any combinations thereof.

6. The liquid pharmaceutical composition of claim 5, wherein said sweetener is selected from the group consisting of maltitol, sucrose, sorbitol, xylitol, mannitol, and any combinations thereof and is present in an amount about 10% to about 85% w/v.

7. The liquid pharmaceutical composition of claim 5, wherein said preservative is selected from the group consisting of methylparaben, propylparaben, sodium benzoate, potassium sorbate, and any combinations thereof and is present in an amount about 0.01% to about 1.0% w/v.

8. The liquid pharmaceutical composition of claim 5, wherein said flavoring agent is present in an amount about 0.001% to about 2% w/v.

9. The liquid pharmaceutical composition of claim 5, wherein said buffering agent is selected from the group consisting of citric acid, sodium citrate, phosphate buffer, acetate buffer, and any combinations thereof and is present in an amount about 0.01% to about 5% w/v.

10. The liquid pharmaceutical composition of claim 5, wherein said pH adjusting agent is selected from the group consisting of dilute acid, dilute base, and any combinations thereof and is present in an amount to adjust said composition pH between about 5 and about 7.

11. The liquid pharmaceutical composition of claim 5, wherein said pharmaceutically active agent other than entecavir is selected from the group consisting of didanosine, lamivudine, abacavir, adefovir, adefovir dipivoxil, famciclovir, (2R,4R)-4-(2,6-diamino-9H-purin-9-yl)-2-hydroxymethyl-1,3-dioxolane (DAPD), hepatitis B immunomodulating proteins (EHT 899 from Enzo Biochem), emtricitabine, 1-(2-deoxy-2-fluoro-β-D-arabinofuranosyl)thymine(FMAU), GLQ-223 (Compound A, alpha-trichosanthin), epavudine (L-dT), epcitabine (L-dC), ribavirin, tenofovir (PMPA), 2′,3′-dideoxy-2′,3′-didehydro-beta-L(-)-5-fluorocytidine[L(-)Fd4C], fluoro L- and D-nucleosides, immunomodulators, and any combinations thereof.

12. The liquid pharmaceutical composition of claim 1, wherein said pharmaceutically acceptable solvent is selected from the group consisting of water, PEG 400, propylene glycol, ethanol, glycerin, and any combinations thereof.

13. The liquid pharmaceutical composition of claim 1, wherein said pharmaceutically acceptable solvent is water.

14. A powder for constitution at the time of use as a liquid pharmaceutical composition to treat hepatitis B virus infection comprising about 0.001 wt. % to about 20 wt. % of entecavir, based on the total weight of the powder composition.

15. The powder composition of claim 14, wherein said entecavir is present in the powder composition in an amount of about 0.003 wt. % to about 10 wt. % based on the total weight of the powder composition.

16. The powder composition of claim 15, wherein said entecavir is present in the powder composition in an amount of about 0.005 wt. % to about 5 wt. % based on the total weight of the powder composition.

17. The powder composition of claim 16, wherein said entecavir is present in the powder composition in an amount of about 0.005 wt. % to about 1 wt. % based on the total weight of the powder composition.

18. The powder composition of claim 14, further comprising at least one component selected from the group consisting of sweetener, preservative, flavoring agent, buffering agent, pH adjusting agent, pharmaceutically active agent other than entecavir, and any combinations thereof.

19. The powder composition of claim 18, wherein said sweetener is selected from the group consisting of sucrose, glucose, acesulfame, dextrose, sorbitol, xylitol, mannitol, and any combinations thereof and is present in an amount about 30 wt. % to about 98 wt. %.

20. The powder composition of claim 18, wherein said preservative is selected from the group consisting of methylparaben, propylparaben, sodium benzoate, potassium sorbate, and any combinations thereof and is present in an amount about 0.01 wt. % to about 5 wt. %.

21. The powder composition of claim 18, wherein said flavoring agent is present in an amount about 0.001 wt. % to about 1 wt. %.

22. The powder composition of claim 18, wherein said buffering agent is selected from the group consisting of citric acid, sodium citrate, phosphate buffer, acetate buffer, and any combinations thereof and is present in an amount about 1 wt. % to about 20 wt. %.

23. The powder composition of claim 18, wherein said pharmaceutically active agent other than entecavir is selected from the group consisting of didanosine, lamivudine, abacavir, adefovir, adefovir dipivoxil, famciclovir, (2R,4R)-4-(2,6-diamino-9H-purin-9-yl)-2-hydroxymethyl-1,3-dioxolane (DAPD), hepatitis B immunomodulating proteins (EHT 899 from Enzo Biochem), emtricitabine, 1-(2-deoxy-2-fluoro-β-D-arabinofuranosyl)thymine(FMAU), GLQ-223 (Compound A, alpha-trichosanthin), epavudine (L-dT), epcitabine (L-dC), ribavirin, tenofovir (PMPA), 2′,3′-dideoxy-2′,3′-didehydro-beta-L(-)-5-fluorocytidine[L(-)Fd4C], fluoro L- and D-nucleosides, immunomodulators, and any combinations thereof.

24. A method of preparing a liquid pharmaceutical composition for oral administration containing a low dose of entecavir comprising the step of dissolving said entecavir and preservative in a solution comprising a pharmaceutically acceptable solvent, wherein said entecavir is present in said solution in an amount about 0.001% to about 20% on a weight/volume basis of the solution.

25. The method of claim 24, wherein said pharmaceutically acceptable solvent is water.

26. The method of claim 24, further comprising the steps of:
(a) dissolving a sweetener in said solution;
(b) dissolving a buffering agent in said solution; and
(c) dissolving a flavoring agent in said solution.

27. A method of preparing a powder for reconstitution at the time of use as a liquid pharmaceutical composition for oral administration containing a low dose of entecavir comprising the step of mixing entecavir with at least one additional component selected from the group consisting of sweetener, preservative, flavoring agent, buffering agent, and any combinations thereof, wherein the powder for constitution is formed and wherein said entecavir is present in said powder for constitution composition in an amount about 0.001 wt. % to about 20 wt. %.

28. A liquid pharmaceutical composition comprising:
about 0.001% to about 20% entecavir;
about 10% to about 85% sweetener;
about 0.001% to about 0.1% flavoring agent;
about 0.01% to about 1% preservative;
about 0.01% to about 5% buffering agent; and
q.s. of pharmaceutically acceptable solvent,
wherein the percentages are on a weight/volume basis.

29. The liquid pharmaceutical composition of claim 28, wherein said entecavir is present in an amount about 0.003% to about 10% w/v.

30. The liquid pharmaceutical composition of claim 29, wherein said entecavir is present in an amount about 0.005% to about 5% w/v.

31. The liquid pharmaceutical composition of claim 30, wherein said entecavir is present in an amount about 0.005% to about 1% w/v.

32. The liquid pharmaceutical composition of claim 28, wherein said entecavir is present in an amount about 0.005% w/v.

33. The liquid pharmaceutical composition of claim 28, wherein said entecavir is present in an amount about 0.02% w/v.

34. A powder for constitution at the time of use as a liquid pharmaceutical composition comprising:
about 0.001 wt. % to about 20 wt. % entecavir;
about 70 wt. % to about 90 wt. % sweetener;
about 0.001 wt. % to about 1 wt. % flavoring agent;
about 0.01 wt. % to about 5 wt. % preservative; and
about 1 wt. % to about 20 wt. % buffering agent,
wherein the percentages are based on the total weight of said powder composition.

35. The powder composition of claim 34, wherein said entecavir is present in an amount about 0.003 wt. % to about 10 wt. % based on the total weight of said powder composition.

36. The powder composition of claim 35, wherein said entecavir is present in an amount about 0.005 wt. % to about 5 wt. % based on the total weight of said powder composition.

37. The powder composition of claim 36, wherein said entecavir is present in an amount about 0.005 wt. % to about 1 wt. % based on the total weight of said powder composition.

38. The powder composition of claim 34, wherein said entecavir is present in an amount about 0.013 wt. % based on the total weight of said powder composition.

39. The powder composition of claim 34, wherein said entecavir is present in an amount about 0.05 wt. % based on the total weight of said powder composition.

40. The powder composition of claim 34, wherein said entecavir is present in an amount about 0.11 wt. % based on the total weight of said powder composition.
